(19) United States
(12) Patent Application Publication
(10) Pub. No.: US 2017/0231482 A1
(43) Pub. Date: Aug. 17, 2017

(54) Sheathed Speculum

(71) Applicant: Pelican Feminine Healthcare Ltd., Llanishen, Cardiff, South Glamorgan (GB)

(72) Inventor: Paul EAKIN, Newtownards (IE)

(73) Assignee: Pelican Feminine Healthcare Ltd., Llanishen, Cardiff, South Glamorgan (GB)

(21) Appl. No.: 15/518,845

(22) PCT Filed: Oct. 12, 2015
(86) PCT No.: PCT/GB2015/052976

§ 371 Date: Apr. 13, 2017

(30) Foreign Application Priority Data
Oct. 16, 2014 (GB) 1418339.6

(51) Int. Cl. A61B 1/32; A61B 1/303; A61B 1/00

(52) U.S. Cl. CPC A61B 1/32; A61B 1/00135; A61B 1/303

(57) ABSTRACT

A speculum is provided, comprising two or more blades moveable between an open and a closed position. A polyisoprene or silicone tubular sheath is attached to at least one of the blades, the sheath being arranged to retain the lateral walls of the body cavity when the blades are in the open position. Sheath attachment means provided on one of the speculum blades facilitate attachment of the sheath to the blade, the attachment means comprising at least one patch or strip of adhesive material extending in a longitudinal direction of the blade.

### CROSS-REFERENCE TO RELATED APPLICATION(S)

This application is the National Stage of International Patent Application No. PCT/GB2015/052976 filed on Oct. 12, 2015 and claims priority from GB Patent Appl. No. 1418339.6, filed on Oct. 16, 2014, both of which are hereby incorporated by reference herein in their entireties.

### BACKGROUND

1. Field

This invention relates to the field of medical tools, in particular to specula.

2. State of the Art

A speculum is a commonly-used medical tool for opening or distending an orifice or cavity of a patient to permit examination of the interior and/or to enable a sample to be taken.

Various forms of specula are known, their designs varying in accordance with the body cavity to be inspected (e.g. vagina, rectum, ear, nostril). However, all forms of specula incorporate the same basic concept: elongate separator elements (paddles′, or ‘blades’), typically having a curved cross section, are positioned edge to edge so that in combination they form an elongate channel. The channel narrows towards the end to be inserted into the orifice (the distal end) to aid the insertion process whilst remaining relatively wider at the opposing (proximal) end to facilitate visual examination or insertion of an instrument via the mouth of the speculum. The blades each have a longitudinal axis extending between the proximal and distal ends.

The two blades are hinged at their broader ends, thus resembling a duck's beak. Thus, the speculum is able to move to move between a closed configuration (i.e. blade edges are adjacent to each other) and an open configuration (i.e. blades are angled away from each other). Handles are attached, usually integrally, at the hinged ends of the blades. Squeezing of the handles towards each other causes the blades to move apart thus dilating the orifice or cavity and enabling examination. When the examination is complete, the handles are moved apart from each other, thus allowing the blades to move towards each other and into the closed configuration such that the speculum can be withdrawn from the cavity.

It is not uncommon for excess tissue to be present in the cavity. In the case of the vagina, for example, women who have had several pregnancies, a prolapsed uterus or other uterine or vaginal damage may have excess vaginal tissue. Obese women may also have an increase in the loose connective tissue beneath the pelvic peritoneum.

In such cases, when such patients have a vaginal speculum examination, the loose vaginal walls may collapse inwards (i.e. towards the midline of the speculum blades) as the blades attempt to keep the anterior and posterior walls apart. The collapse of the vaginal walls results in an obscuring of the visualisation of the cervix for the purpose of cervical cultures, ruptured membranes, pap smears, biopsy or the visual assessment of the degree of dilation.

The problem of vaginal wall collapse has been addressed in known specula. For example, U.S. Pat. No. 6,432,048 discloses a speculum having a flexible sheath which is configured to expand during opening of the blades but collapse into a folded configuration when the blades are closed, the folded sheath being held between the closed blades. Thus, when the speculum is pushed into the cavity, the sheath may be pushed towards the mouth of the speculum, and so pockets are provided around the distal tips of the blades to prevent such movement. Moreover, upon withdrawal of the speculum, the sheath may slide off the blades unless the blades are closed sufficiently tightly as to clamp the folded sheath therebetween.

GB 2424585 discloses a speculum having a flexible wall member attached to the distal end of at least one of the blades by means of adhesive. When the blades are opened the wall member expands. It then retains this expanded shape when the blades are subsequently closed. Thus, the wall member does not return to its original formation and remains in an expanded or stretched state. Upon withdrawal of the speculum, the wall member is retracted back along the blades by friction contact with the body cavity.

U.S. Pat. No. 6,902,530 discloses an elastic mesh covering for a speculum, the cover being attached to the speculum by means of strings which the practitioner must tie around the back of the device before use and untie after use. The strings may be fiddly to use, may obscure the practitioner's view during use, and may hinder the use of instruments at the mouth of the speculum. Moreover, the cover extends over the length of both blades and partially obscures the practitioner's view via the open distal ends.

U.S. Pat. No. 5,787,648 also shows a sheath for use with a speculum. However, the sheath is not attached to the blades and also retains its shape when the blades are closed and partially withdrawn. Similarly, U.S. Pat. No. 6,036,638 shows a speculum sleeve which is not attached to the speculum.

US 2005/0192482 A discloses a sheathed speculum wherein the sheath is attached to the speculum via a ‘coating’ of adhesive. However, coating the sheath requires a significant amount of adhesive, which in turn is costly.

US 2008/0242938 A1, U.S. Pat. No. 6,432,048 B1, U.S. Pat. No. 3,841,317, US 2014/0163322 A1, US 2004/0186355 A1 and US 2003/0069477 A1 all disclose known arrangements for attaching a sheath or membrane to a speculum blade by means of adhesive.

GB patent document 2482148 A discloses a speculum having a polyurethane sheath attached to the speculum using a strip of adhesive. However, it has been found that in use a polyurethane sheath does not provide significance stretch and flexibility and requires a significant degree of force to open the speculum blades against the urging force of the sheath. The excess force required to open the speculum can result in breakage of the blades. This poses a safety concern.

### SUMMARY

An improved arrangement has now been devised. In accordance with the invention a solution is provided as defined in the appended claims.

According to the invention there is provided a speculum comprising

two or more blades moveable between an open and a closed position;

a tubular sheath comprising polyisoprene or silicone; and

an elongate area of adhesive for attaching the sheath to at least one of the blades.

Thus, the sheath is made using a material which contains, partially or entirely, either polyisoprene or silicone. The silicone sheath may comprise liquid silicone rubber (LSR) or high consistency silicone rubber (HCR). Such a sheath provides several advantages over known sheaths. For example, the use of polyisoprene or silicone provides a more flexible and stretchable sheath which does not exert excess force on the speculum when opening the blades while still providing sufficient strength to hold back the wall of the cavity. It is also comfortable for the patient in use. Polyisoprene may be preferred due to its lower cost compared to silicone.

Preferably, the sheath is dip moulded. This avoids disadvantages arising from other known techniques such as welding.

Preferably, the width of the elongate area of adhesive is less than 10 mm. In a preferred embodiment, the elongate area of adhesive is in the range of 5 to 8 mm wide. In some embodiments, the width may be 6 mm. This provides the advantage that the adhesive provides a sufficiently secure attachment of the sheath to the speculum blade, whilst allowing a substantial surface area of the sheath to remain unattached. The more surface area of sheath attached to the blade, the less the sheath is able to stretch freely during use. This degrades performance of the sheath and makes it harder to open the blades during use. The use of silicone or polyisoprene for the sheath enables the adhesive strip to be narrower than prior art arrangements.

The thickness of the sheath may be greater than 50 microns. Preferably, the thickness is greater than 100 microns. In a preferred embodiment it may be 150 to 300 microns. A thickness towards the lower end of this range may be preferred. The thickness may be 150 microns. A sheath of such thickness has been found to provide enhanced resilience to punctures, piercing and breakage compared to prior art sheaths.

Preferably, the adhesive area is a strip (which may comprise a series of adhesive patches or blobs) which extends in a longitudinal direction of the blade (i.e., in a direction aligned with the longitudinal axis of the blade) running from the end towards mouth to the opposite (tip) end of the blade. The adhesive area may comprise a spot weld. It may comprise impulse welding, ultrasonic welding or thermal welding. It may comprise any material or technique used to adhere the sheath to the speculum blade, such as glue. The adhesive may be chosen due to its effectiveness with polyisoprene and/or silicon.

The adhesive area may comprise double sided PET tape. The double coated tape may comprise a 20 μm transparent polyester film. It may be coated on both sides with a hypoallergenic, tackified synthetic rubber-based adhesive. It may comprise a paper release liner. It may be an adhesive product such as available as of October 2014 via manufacturer 3M, product number 9877.

The adhesive area may comprise high tack transfer adhesive. It may be Hi-Tack medical transfer adhesive comprising a synthetic rubber adhesive. It may also comprise a release paper liner. It may be an adhesive product such as available as of October 2014 via manufacturer 3M, product number 1504.

The adhesive area may extend along or in line with the longitudinal axis of the blade and/or sheath. The adhesive area may be spaced from at least one of the terminal edges of the sheath such that there is a non-adhesive margin at the proximal and/or distal end of the sheath. Preferably, the margin between the adhesive area and the edge of the sheath is minimal such as 0.5 mm or thereabouts. Alternatively, the adhesive area may extend along the entire longitudinal length of the sheath. Thus, the adhesive may extend to one or both terminal edges of the sheath or close to the edge(s). This provides the advantage that the sheath is less likely to be rolled back along the speculum during insertion into the body, or pulled off during removal.

The sheath may be substantially circular in cross section. Alternatively or additionally, the sheath may taper in diameter towards one end such that it is narrower at that end. However, the sheath may, in some embodiments, have a constant diameter along its longitudinal length. Preferably, the sheath is open at both ends to enable insertion of the speculum blades through both open ends of the sheath.

In a preferred embodiment, the sheath is open-ended and covers the speculum by encircling an area of the blades between their distal and proximal ends. Preferably, the sheath encircles a mid-section of the blades such that it extends along only a portion of the blades lengths. Thus, the sheath may not extend along the entire length of the blade. Alternatively, the sheath may cover substantially the entire length of the blades.

The sheath may be removably or permanently attached to the speculum.

Preferably, the area of adhesive material is provided towards or on the midline of the speculum blade, such as the apex of a curved speculum blade. Preferably, the adhesive material is provided on the outer surface of a blade to facilitate attachment of the sheath. Thus, in use, the adhesive material sits between the sheath and the blade, holding the sheath in secure relation to the speculum. The adhesive strip provides the advantage that whilst providing a secure attachment of the sheath to the speculum, it is also relatively flat or shallow in profile and so does not stand proud of the blade to any great degree. Thus avoids causing damage to the cavity tissue or discomfort to the patient.

The adhesive strip may not extend width-wise to the longitudinal edges of the speculum blades. Thus, the area of sheath/blade around the adhesive strip may not be exposed to the adhesive material. This provides the advantage that only a relatively small amount of adhesive is required to achieve a secure attachment to the blade, thus lowering manufacturing costs. This is a significant improvement over arrangements which coat the sheath or blade in adhesive, and thus are more costly.

Preferably, the adhesive strip is provided with a removable protective member. This protective member may be a peelable cover or shield which is provided over the adhesive prior to application of the sheath in order to protect the adhesive from damage and/or prevent accidental adhesion of to the sheath whilst the sheath is being positioned on the speculum. The protective member may be folded over upon itself substantially in a U-shape. One arm of the U may be longer than the other to facilitate gripping of the arm such that a user can grasp the protective member and peel it away from the adhesive. The sheath can be positioned on the speculum with the protective member in place, and the protective member can then be peeled back in order to allow the adhesive present on the speculum to contact and secure the sheath. This provides the advantage that the sheath can be applied to the speculum with ease, thus facilitating assembly of the sheath and speculum.

The adhesive may be such that it can withstand multiple uses i.e. the sheath may be applied to the blade more than once, and/or the peel-off strip may be re-applied more than once, without the attachment means losing its adhesive qualities.

In some embodiments of the invention, the speculum may be provided with the sheath already attached to the speculum prior to use by a practitioner. Alternatively, the adhesive strip and sheath are provided separately from the speculum for assembly by a practitioner prior to use on a patient.

The adhesive area may be a double-sided adhesive strip wherein the first side of the adhesive strip is configured for adhesion to the speculum blade and the second side is configured for adhesion to the sheath.

A plurality of adhesive areas may be provided. Preferably, an elongate adhesive strip may be provided on each blade. This provides a more secure attachment and reduces or eliminates the possibility of the sheath becoming unattached from the speculum during use. It also provides the advantage that the sheath is less likely to tear, slip, wrinkle or bunch up during insertion or withdrawal of the speculum from the body because both the top and bottom of the sheath are held in position on the blades.

Preferably, the sheath is substantially transparent. Thus, the practitioner's view of the cavity walls is not impeded by the sheath when using it with a transparent (e.g. plastic) speculum. Preferably, the sheath is a solid tube or band. In some embodiments it may have recesses or apertures formed in the body of the sheath. The sheath may be a mesh.

Preferably, the sheath is biased to an unexpanded, initial configuration (e.g. shape and size) which it maintains when the speculum blades are closed, but is stretched into an expanded configuration when the speculum blades move away from one another into an open position. The speculum blades may push against the resilience of the sheath as the blades open. As the blades return from an open to a closed position, the sheath contracts such that it returns to and retains its original, unexpanded form. Thus, the sheath does not collapse, balloon or sag after the blades have been opened.

This provides the advantage that the contracted sheath does not interfere with or collapse between the blades when they are closed. Furthermore, if the blades are re-opened the sheath may maintain substantially its original resilience and continue to urge the blades back towards each other as it did upon first use.

Also according to the invention there is provided a method of manufacturing a speculum sheath comprising silicone or polyisoprene, the method comprising the step of forming the sheath using a dip moulding process.

The method may comprise the steps:

forming a polyisoprene or silicone sheath using a dip moulding process; and

providing at least one elongate area of adhesive on at least one of the speculum's blades; and

bringing the sheath into contact with the adhesive to attach the sheath to the speculum.

The method may further comprise the step of applying soapy water to the sheath prior to attaching it to the speculum. This provides the advantage that residue is removed from the sheath, enabling it to glide over the speculum blades without sticking or adhering to the speculum. This facilitates assembly of the sheathed speculum and reduces the risk of damage to the sheath during application to the speculum.

The thickness of the sheath may be greater than 50 microns. It may be in the range of 150 to 300 microns. Beneficially, it may be around 150 microns. The width of the elongate area of adhesive may be less than 10 mm.

Features described above in relation to one embodiment or aspect of the invention may also be applicable to any other embodiment or aspect of the invention. Thus, features described in relation to the device may also be present in relation to the method and/or vice versa.

Exemplary embodiments of the present invention are described in greater detail below with reference to the drawings which are partially schematic diagrams.

### BRIEF DESCRIPTION OF THE DRAWINGS

FIG. 1 is a side view of a typical speculum, the speculum being in a closed configuration.

FIG. 2 shows a side view of a speculum in accordance with a preferred embodiment of the invention, the speculum being in the closed position and the sheath attached to both the top and the bottom blades.

FIG. 3 shows the speculum of FIG. 2 in the open position.

FIG. 4 shows an alternative embodiment of the invention in which the sheath is attached only to the top blade of the speculum.

FIG. 5 shows an alternative embodiment of the invention in which the sheath is attached only to the bottom blade of the speculum.

FIG. 6 shows a detailed view of the adhesive strip, protective shield and sheath provided on the speculum in accordance with an embodiment of the invention.

FIG. 7 shows a plan view of a speculum in accordance with the present invention.

### DETAILED DESCRIPTION OF THE PREFERRED EMBODIMENTS

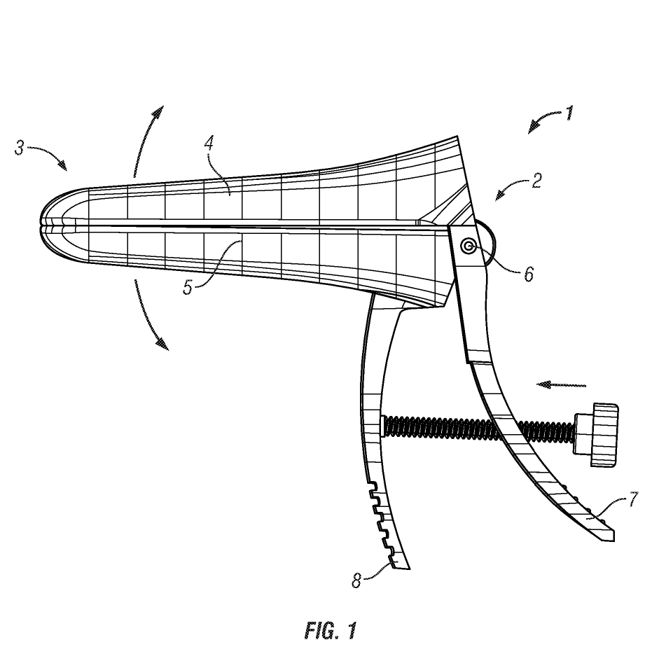

Referring initially to FIG. 1, there is shown a vaginal speculum (1) of the type commonly used for dilating the vaginal cavity or orifice of a patient during examination or medical procedure. Such specula are typically manufactured from a plastic or metal material, having a plurality of curved separator elements typically referred to as ‘blades’ or ‘paddles’. In a typical arrangement, there are two blades (4, 5) oriented such that one provides an upper blade (4) and the other provides a lower blade (5).

The end of the speculum which will be closest to the practitioner during use of the speculum is typically open and known hereafter as the ‘proximal end’ or ‘mouth’ (2). The body cavity may be inspected through the open proximal end or mouth (2). The other end of the speculum, which is inserted into the cavity, is known hereafter as the ‘distal end’ (3). A longitudinal axis extends between the proximal and distal ends of each blade.

The blades (4, 5) are coupled such that they can move towards or away from each other. Depending on the coupling arrangement used, this may be angular or lateral movement. In a typical arrangement, as shown in FIG. 1, the coupling is a hinge (6) provided at the proximal end (2) of the speculum (1), such that the blades (4, 5) may pivot around the hinge (6) at their proximal ends (2) thus moving the distal ends (3) away from or towards each other. The open mouth (2) formed at the proximal end of the blades enables a practitioner to insert an examination or surgical device into the speculum through the mouth, and also enabling visual inspection of the cavity or body part.

Thus, the speculum 1 is moveable between a closed configuration (as shown in FIG. 1) or in an open (expanded) configuration in which there is angular separation displacement of the blades (4, 5).

The blades (4, 5) are provided, coupled integrally or otherwise, with handles (7, 8). Upper blade (4) is shown as coupled to rear handle (7), and lower blade (8) is coupled to front handle (8). Pivoting the handles (7, 8) together thus causes the blades (4, 5) to move apart into an open (expanded) configuration.

It should be noted that a number of different mechanisms exist for supporting the blades and the present invention is not limited in this regard. The present invention may be used in conjunction with a variety of types or styles of speculum.

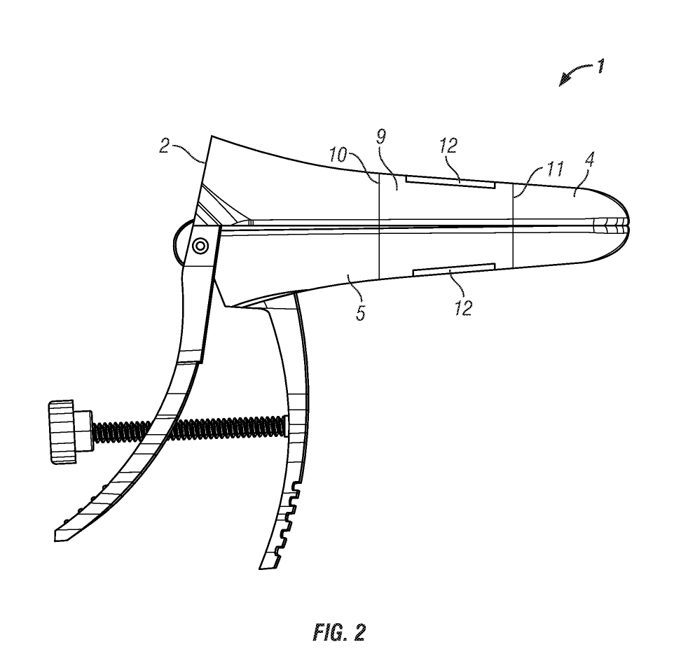

Turning to FIGS. 2 to 7, a speculum is provided according to the invention and has a flexible sheath (9) wrapped around a portion of the blades (4, 5). The sheath is a polyisoprene or silicone sheath which is generally tubular in shape, and formed by dip moulding. The thickness of the sheath is approximately 150 microns.

The sheath (9) has two ends: a proximal (10) end which, in use, is oriented towards the proximal end of the blades 2; and a distal end (11) which, in use, is oriented towards the distal end of the blades (3). A longitudinal axis extends between the opposing (distal and proximal) ends of the sheath. Both ends (10, 11) of the tubular sheath (9) are open to enable the speculum blades to pass through both ends. The distal end 11 of the sheath (9) has a slightly smaller diameter than that of the proximal end (10), such that the sheath (9) is tapered. The nature of the elastic sheath (9) is such that the sheath conforms to and clings to the speculum (1) in a skin-like fashion.

When the speculum 1 is closed as shown in FIG. 2, the sheath (9) is in its initial, unexpanded form. The sheath may be arranged and configured to resiliently bias the blades (4, 5) towards the closed position, although the urging force and resilience of the sheath may vary according to a variety of factors, such as dimensions, material and so on. The sheath (9) is transparent or semi-transparent such that visualisation of the cavity is not impeded during use.

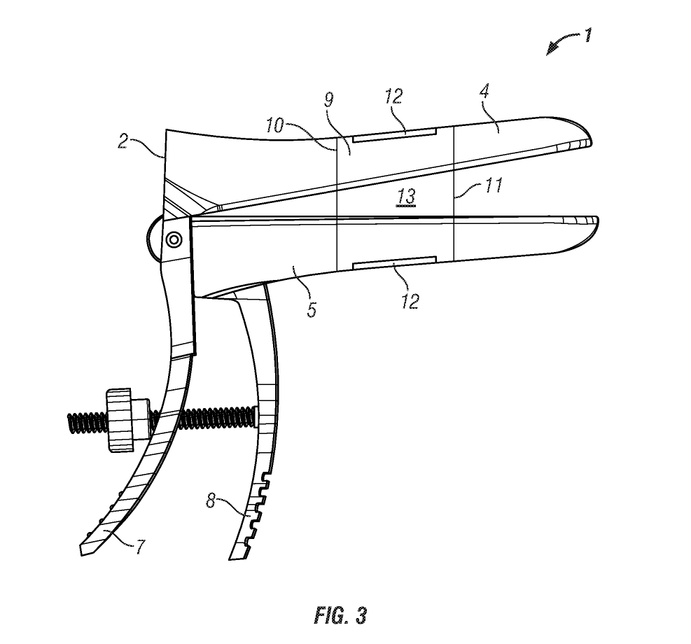

FIGS. 3, 4 and 5 show the speculum (1) in an open configuration. With the speculum in this position, the sheath (9) is stretched into an expanded form by the blades. Thus, the diameter of the sheath has increased at the distal end relative to its diameter when in its initial, contracted form. In this position, the resilient sheath may apply a force to the blades to urge them towards the closed position of FIG. 2.

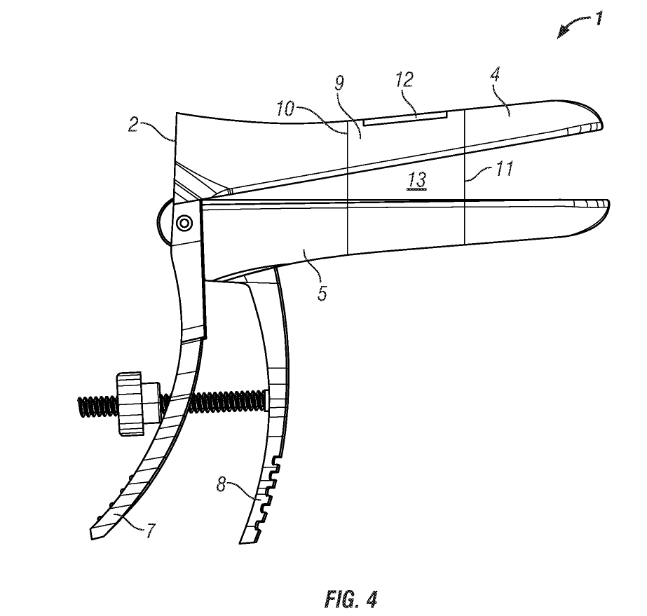

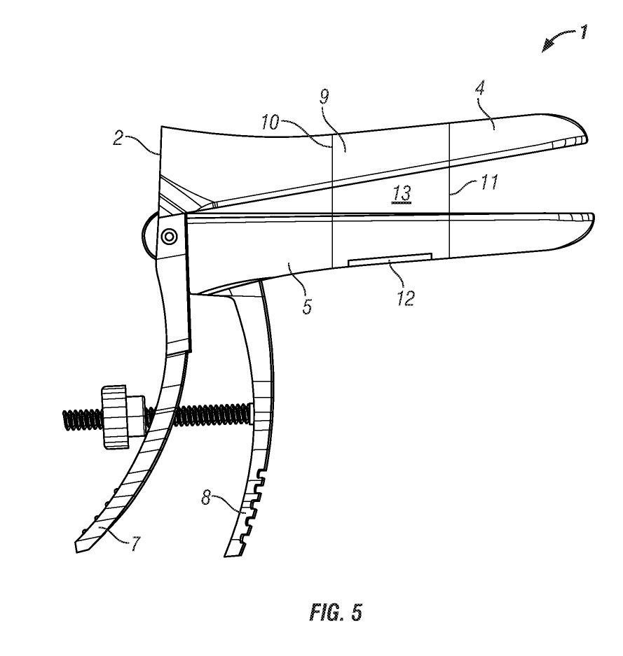

The sheath (9) is attached to or carried on at least one blade. FIGS. 2 and 3 show the sheath attached to the external surfaces of both the top and bottom blades (4, 5). However, alternative embodiments are shown in FIGS. 4 and 5 which show the sheath (9) attached to only the top (4) and bottom (5) blades respectively. The embodiment of FIGS. 2 and 3 is generally preferred because the attachment to both blades provides a more secure attachment of the sheath and reduces the likelihood that the sheath will rip, wrinkle or bunch up or detach from the speculum (1) during insertion/withdrawal into/from the body cavity.

The thickness of the sheath is greater than 50 microns, although a thickness in the region of 150 to 300 microns may be preferably. A thickness of 150 microns has been found to be particularly advantageous in reducing the likelihood of damage whilst retaining comfort for the patient and stretching properties.

The sheath (9) is attached to the blades by an adhesive strip (12) as shown in the Figures although other types of attachment means may be used to similar effect. The strip (12) is an elongate area of adhesive. However, other forms or shapes may be used. For example, a blob or dot of adhesive (or series of spaced blobs or dots of adhesive) may be used instead of an elongate, generally rectangular strip. However, the elongate strip (12) provides enhanced hold of the sheath (9) due to its configuration and shape. Advantageous adhesive materials have been found to include rubber based adhesives, double sided PET tape and high tack transfer adhesive because these work well with the polyisoprene/silicon material of the sheath.

The strip (12) extends in the longitudinal direction of the blade and/or sheath. If being used with a speculum having curved blades, the adhesive strip may be provided at the apex of the curve.

FIG. 7 shows an adhesive strip which is spaced from the terminal edges of the sheath 10, 11 such that a narrow adhesive-free margin is provided at the edges of the sheath. Preferably, the margin is as narrow as possible e.g. about 0.5 mm. Thus, the adhesive strip extends substantially the length of the sheath, from one terminal edge to the other. This prevents rolling back of the sheath during insertion, and reduces the likelihood of the sheath being retained in the body during removal. In another embodiment the adhesive extends the entire length of the sheath.

The adhesive strip does not extend width-wise to the longitudinal edges of the speculum blades. Thus, the area of sheath adjacent the longitudinal side of the adhesive strip is not attached to the speculum, there being a non-adhered margin around the strip which is not in contact with the adhesive. Ideally the strip is as narrow as possible whilst still providing a secure attachment of the sheath. The narrower the strip of adhesive, the more freely the sheath is able to stretch during use. This freedom to stretch provides an enhanced performance because it reduces the force required to open the blades, thus reducing the likelihood of breakage.

The sheath may be removably attached to the speculum. In such embodiments, the adhesive is selected such that the flexible sheath can be detached and possibly re-attached (for example, for re-positioning of the sheath). In other embodiments, the sheath may only be attached to the speculum once, such that removal and/or re-attachment are not possible.

During assembly or manufacture, the adhesive strip (12) is attached to an external surface of the speculum blade (4). This may be performed during manufacture of the speculum, such that the speculum is already provided with the adhesive strip in position on the speculum blade. Alternatively, a double-sided adhesive strip may be provided separately from the speculum such that one side of the adhesive strip is applied to a speculum blade prior to fitting the sheath around the speculum on top of the other side of the adhesive strip.

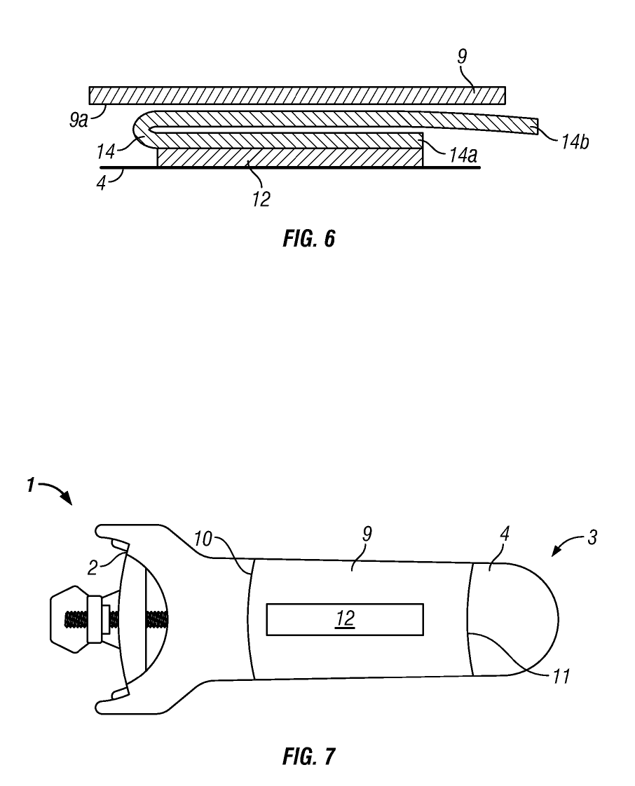

The adhesive strip (12) is protected by a substantially U-shaped, peel-off shield (14) which is folded over onto itself, having two arms (14 a, 14b). This is shown in FIG. 6. The first arm (14a) of the U is applied to the adhesive strip to cover it. Thus, the adhesive strip is not exposed and cannot stick to the inner surface (9a) of sheath (9) during application of the sheath. The second arm (14b) rests on top of the first arm (14a). The second arm (14b) is of sufficient length to extend beyond the edge of the sheath when the sheath is applied, to enable the second arm (14b) to be easily accessible.

The sheath (9) is then fitted around the speculum in the desired position. The second arm (14b) of the shield may then be gripped and pulled to peel the shield away from the adhesive strip (12), exposing it. The inner surface (9a) of the sheath is then brought into contact with the adhesive to attach the sheath securely to the speculum blade.

In cases where a double sided adhesive strip is used, a protective shield can be provided for each adhesive surface of the strip. The protective shield (14) is peeled away from one side of the adhesive strip, and the strip is attached to the blade prior to peeling off the protective shield from the second side of the strip. The exposed second side of the adhesive strip then adheres to the internal surface of the sheath when the sheath (9) is fitted around the speculum (1). Thus, the adhesive strip is sandwiched between the sheath and the speculum blade. Assembly of the sheathed speculum may be performed by the manufacturer or by a practitioner.

During use (i.e. after manufacture and/or assembly as described above) the distal end (3) of the closed, sheathed speculum shown in FIG. 2 is inserted into the body cavity. The blades (4, 5) are then urged apart to open the speculum as per FIGS. 3, 4 and 5. As the blades open, they push against and retain the cavity walls to enable examination. During this process, the sheath (9) is stretched by the opening blades, thus providing two retaining walls or barriers (13) on either side of the speculum between the edges of the blades. The stretched sheath (9) prevents the cavity walls from collapsing into the speculum between the open blades so that the examination or medical procedure may be carried out without impediment, as most clearly seen in FIGS. 3, 4 and 5.

After the examination is complete, the practitioner allows the blades (4, 5) to return to their initial, closed position (as per FIG. 2) prior to withdrawing the speculum (1). As the blades close, the elastic sheath (9) contracts back to its original form and size. Thus, the sheath (9) is biased towards its initial, un-stretched configuration. Thus, sagging or ballooning of the sheath (9) is eliminated or at least minimised.

After use, the sheath (9) may be disposed of along with a disposable speculum. Alternatively, if the sheath is used with a non-disposable speculum, it may be removed and discarded. The non-disposable speculum may then be sterilised prior to re-use.

The polyisoprene or silicone sheath is made using a dip moulding process. Prior art techniques include welding the sheath together using ultrasonic welding. However, due to the fragile nature of the material and the force required to open the speculum such sheaths are liable to break during assembly or use. The dip moulding process eliminates the potential fragile weld issue and allows the adhesive strip size to be reduced to a minimum amount, thus allowing the maximum surface area of the sheath material to be left un-adhered to the speculum allowing for maximum stretch. This, in turn, reduces the force exerted on the speculum handles.

Prior to applying the sheath to the speculum, the sheath is rinsed or immersed in soapy water. This removes residue from the surface of the sheath and enables the sheath to glide over the surface of the speculum without sticking to it. This facilitates the assembly process.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. In the present specification, “comprises” means “includes or consists of” and “comprising” means “including or consisting of”. The singular reference of an element does not exclude the plural reference of such elements and vice-versa. In a device claim enumerating several means, several of these means may be embodied by one and the same item of hardware. The mere fact that certain measures are recited in mutually different dependent claims does not indicate that a combination of these measures cannot be used to advantage.

1. A speculum comprising
two or more blades moveable between an open and a closed position;
a tubular sheath comprising polyisoprene or silicone; and
an elongate area of adhesive for attaching the sheath to at least one of the blades.

2. A speculum according to claim 1, wherein the sheath is a dip-molded sheath.

3. A speculum according to claim 1, wherein the width of the elongate area of adhesive is less than 10 mm.

4. A speculum according to claim 1, wherein the sheath is greater than 50 microns in thickness.

5. A speculum according to claim 1, wherein the elongate area of adhesive extends in a longitudinal direction of the blade.

6. A speculum according to claim 1, wherein the elongate area of adhesive is provided with a removable protective member.

7. A speculum according to claim 6, wherein the protective member is substantially U-shaped.

8. A speculum according to claim 1, wherein the elongate area of adhesive is spaced from a peripheral feature of the speculum blade such that a non-adhered margin is formed around at least a portion of the attachment means.

9. A speculum according to claim 1, wherein the elongate area of adhesive comprises a double-sided adhesive strip, the first side of the adhesive strip being configured for adhesion to the speculum blade and the second side being configured for adhesion to the sheath.

10. A speculum according to claim 1, wherein the elongate area of adhesive comprises a plurality of spaced blobs of adhesive material, or a spot weld comprising impulse welding, ultrasonic welding or thermal welding.

11. A method of manufacturing a speculum, comprising:
forming a polyisoprene or silicone sheath using a dip-molding process; and
providing at least one elongate area of adhesive on at least one of the speculum's blades; and
bringing the sheath into contact with the adhesive to attach the sheath to the speculum.

12. A method according to claim 11, further comprising applying soapy water to the sheath prior to attaching it to the speculum.

13. A method according to claim 11, wherein the sheath greater than 50 microns.

14. A method according to claim 11, wherein the elongate area of adhesive has a width less than 10 mm.
